(19) United States
(12) Patent Application Publication
(10) Pub. No.: US 2013/0066059 A1
(43) Pub. Date: Mar. 14, 2013

(54) CYCLIZING LINKER AND CYCLIC CAGED MORPHOLINOS

(71) Applicants: JAMES K. CHEN, Mountain View, CA (US); ILYA A. SHESTOPALOV, Inverness, IL (US); SAYUMI YAMAZOE, Palo Alto, CA (US)

(72) Inventors: JAMES K. CHEN, Mountain View, CA (US); ILYA A. SHESTOPALOV, Inverness, IL (US); SAYUMI YAMAZOE, Palo Alto, CA (US)

(73) Assignee: THE BOARD OF TRUSTEES OF THE LELAND STANFORD JUNIOR UNIVERSITY, Palo Alto, CA (US)

(21) Appl. No.: 13/593,471

(22) Filed: Aug. 23, 2012

Related U.S. Application Data
(60) Provisional application No. 61/534,319, filed on Sep. 13, 2011.

(51) Int. Cl. C07H 17/00; C07D 207/46

(52) U.S. Cl. 536/24.5; 548/542

(57) ABSTRACT

A bifunctional and photocleavable linker for cyclizing a morpholino-based oligonucleotide, having the formula:

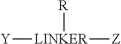

with the Linker as a photoactive group derivatized spacer molecule that presents two functional groups for cyclizing a selected morpholino and with the R group as a photoactive group utilized in the process of photocleavage and the X and Z groups are functional groups utilized for attachment to a morpholino to generate a cyclic structure.

### CROSS-REFERENCE TO RELATED APPLICATIONS

This application claims priority from U.S. provisional patent application Ser. No. 61/534,319 filed on Sep. 13, 2011, incorporated herein by reference in its entirety.

### STATEMENT REGARDING FEDERALLY SPONSORED RESEARCH OR DEVELOPMENT

This invention was made with Government support under Grant No. DP1 OD003792, awarded by National Institutes of Health. The Government has certain rights in this invention.

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

### 1. Field of the Invention

The subject invention pertains generally to the synthesis of caged and cyclized morpholino-based oligonucleotides by means of a photocleavable bifunctional linker, wherein the derivatized morpholinos are employed to inhibit gene expression in cells or organisms with spatial and temporal resolution. The methodology disclosed here is a novel way to cyclize and cage (or temporarily inactive) these anti-sense reagents for in vivo procedures.

### 2. Description of Related Art

Morpholinos (MO) are artificial nucleosides that are typically used as 25-base oligomers to block RNA splicing or translation in cultured cells or whole organisms. Previous strategies for caging these reagents include: (1) tethering a complementary, inhibitory oligomer to the targeting morpholinos via a photocleavable linker, thereby generating a caged hairpin; (2) mixing the morpholino with several molar equivalents of a complementary, synthetic RNA oligonucleotide containing two 12-base oligomers linked by a photocleavable group, thereby forming a caged morpholino/RNA duplex; (3) mixing the morpholino with a 1.1-1.3 molar equivalents of a partially complementary morpholino oligonucleotide containing two 12-base oligomers linked by a photocleavable group, thereby forming a caged morpholino/morpholino duplex; and (4) modifying four or more morpholino bases with photocleavable groups to prevent RNA hybridization.

### 3. References

(1) Shestopalov, I. A.; Sinha, S.; Chen, J. K. Nat. Chem. Biol. 2007, 3, 650-1.
(2) Ouyang, X.; Shestopalov, I. A.; Sinha, S.; Zheng, G.; Pitt, C. L.; Li, W. H.; Olson, A. J.; Chen, J. K. J. Am. Chem. Soc. 2009, 131, 13255-69.

### BRIEF SUMMARY OF THE INVENTION

The present invention generally comprises a bifunctional and photocleavable linker for cyclizing a morpholino-based oligonucleotide, having the formula:

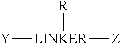

with the Linker as a photoactive group derivatized spacer molecule that presents two functional groups for cyclizing a selected morpholino and with the R group as a photoactive group utilized in the process of photocleavage and the X and Z groups are functional groups utilized for attachment to a morpholino to generate a cyclic structure.

Further aspects and embodiments of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 shows the classification of ntla mutant phenotypes according to morphological cues. Somitic (s), medial floor plate (mfp), notochord (nc), and yolk extension (ye) tissues are labeled.

FIG. 2 presents the distribution of phenotypes for cyclic and hairpin cMOs at a dose of 115 fmol/embryo with and without global UV photoactivation at 3 hours post fertilization (hpf).

FIG. 3 depicts the measurement of Ntla protein levels in 10-hpf embryos injected with a conventional ntla MO, a cyclic ntla cMO, or a hairpin ntla cMO at the one-cell stage and then either cultured in the dark or globally UV irradiated at 3 hpf.

FIG. 4 shows a representative western blot for the cyclic and hairpin cMOs.

FIG. 5 shows the classifications of ptf1a mutant phenotypes according to pancreas-specific eGFP expression in the transgenic zebrafish.

FIG. 6 shows the distribution of phenotypes for cyclic cMOs at a dose of 230 fmol/embryo with and without global UV photoactivation at 3 hours post fertilization (hpf).

FIG. 7 shows the distribution of phenotypes for hairpin cMOs at a dose of 230 fmol/embryo with and without global UV photoactivation at 3 hours post fertilization (hpf).

### DETAILED DESCRIPTION OF THE INVENTION

The subject invention involves the intramolecular cyclization of morpholinos using a photocleavable linker. The cyclized oligonucleotide is conformationally resistant to RNA hybridization, and upon linker photolysis, the liberated linear oligomer regains its activity. Reagent efficacy varies with oligonucleotide length and cyclic morpholino structure.

Morpholinos are effective reagents for gene silencing, however, they act immediately and globally (in the context of organisms such as zebrafish). An ability to control the place and timing of morpholino action is important for gaining spatiotemporal control of gene expression.

Previous strategies for caging morpholinos are synthetically more challenging than the approach utilized with the subject invention. The subject methodology provides rapid access to caged reagents using oligonucleotide precursors that are commercially available.

Various embodiments of the subject invention include, but are not limited to: different targeting morpholino lengths (less than 25 bases); different photocleavable linkers (various lengths and chromophores); addition of self-complementary sequences to the targeting morpholino to generate cyclic hairpins upon cyclization; and replacement of the photocleavable group with one that can be enzymatically cleaved.

It is stressed that the subject linker that is used to cyclize morpholinos is also new. The subject method provides a way for caging morpholinos that is synthetically more accessible than current approaches.

### Examples

### Chemical Systhesis

General Synthetic Procedures.

All reactions were carried out in flame-dried glassware under a nitrogen atmosphere using commercial reagents without further purification, unless otherwise indicated. Reactions were magnetically stirred and monitored by thin layer chromatography (TLC), using glass-backed silica gel 60_{F254} (Merck, 250 μm thickness). Yields refer to chromatographically and spectroscopically pure compounds unless otherwise stated. SiO₂ chromatography was carried out with EM Science silica gel (60 Å, 70-230 mesh) as a stationary phase. ¹H NMR and ¹³C NMR spectra were acquired on Varian 400 MHz spectrometers and standardized to the NMR solvent peak. Electrospray (ESI) mass spectra were obtained using a Micromass ZQ single quadrupole liquid chromatography-mass spectrometer (LC-MS) and a Micromass Q-TOF hybrid quadrupole LC-MS.

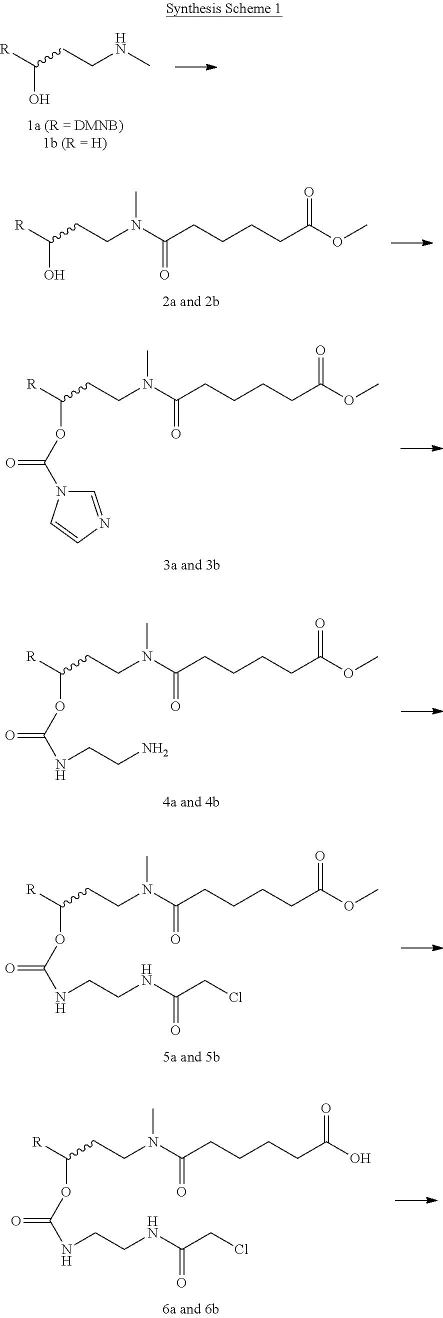

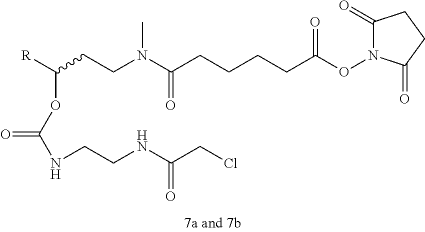

Compound 2a was synthesized according to reported procedure^{1,2} from a commercially available compound, 4,5-dimethoxy-2-nitrobenzaldehyde (DMNB).

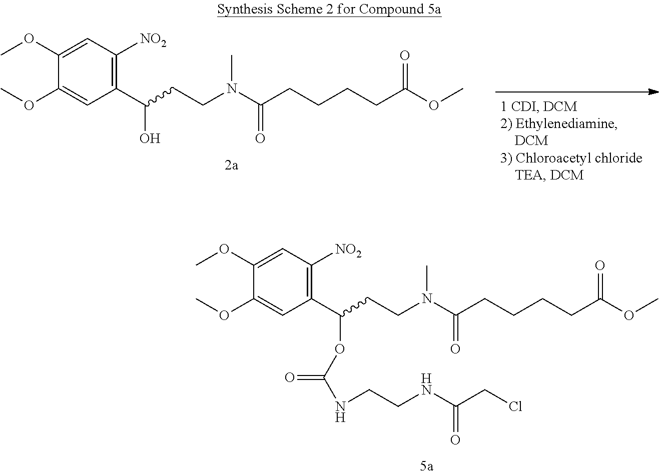

### Methyl 1-chloro-9-(4,5-dimethoxy-2-nitrophenyl)-12-methyl-2,7,13-trioxo-8-oxa-3,6,12-triazaoctadecan-18-oate (5a)

Compound 2a (24.3 mg, 0.0589 mmol) was dissolved in anhydrous DCM (0.750 mL) and added to 1,1′-carbonyl diimidazole (23.9 mg, 0.147 mmol) in anhydrous DCM (1.00 mL). The reaction mixture was stirred for 3 h at room temperature under nitrogen, diluted with CHCl₃, washed two times with water, and dried over anhydrous MgSO₄. Solvent was removed in vacuo to yield crude imidazole carbamate as a yellow gum 3a (27.2 mg, 0.0537 mmol).

The imidazole carbamate 3a (27.2 mg, 0.0537 mmol) was dissolved in anhydrous DCM (300 μL), and the solution was cooled to 0° C. Ethylenediamine (11.7 μL, 0.159 mmol) was added, and the reaction mixture was stirred for 3 h at room temperature under nitrogen. Solvent was removed in vacuo to yield crude amine 4a as a yellow oil (21.4 mg, 0.0429 mmol). MS-ESI (m/z): [M+H]⁺ calculated for C₂₂H₃₅N₄O₉⁺, 499.2; observed, 499.2. [M+Na]⁺ calculated for C₂₂H₃₄N₄NaO₉⁺, 521.2; observed, 521.4. HRMS-ESI (m/z): [M+H]⁺ calculated for C₂₂H₃₅N₄O₉⁺, 499.2399; observed, 499.2392. Without further purification, compound 4a (21.4 mg, 0.0429 mmol) was dissolved in anhydrous DCM (300 μL) and triethylamine (30.0 μL, 0.215 mmol) and cooled to 0° C. 2-Chloroacetyl chloride (6.80 μL, 0.0855 mmol) dissolved in anhydrous DCM (100 μL) was added slowly to this solution. The mixture was allowed to stir at room temperature for 20 min, at which time, 5% saturated aq. NaHCO₃ was added. Organic layer was dried over anhydrous MgSO₄. Solvent was removed in vacuo, and the residue was purified by SiO₂ column chromatography (CHCl₃/acetone, stepwise gradient from 1/0 to 1/1) to yield 5a as a colorless oil (18.6 mg, 54% from 2a). ¹H NMR (400 MHz, CDCl₃) δ 8.18 (m, 1H), 7.61 (br, s, 1H), 7.02 (br, s, 1H), 6.06 (m, 1H), 5.25 (m, 1H), 4.38 (m, 1H), 4.01 (s, 2H), 3.96 (s, 3H), 3.93 (s, 3H), 3.66 (s, 3H), 3.52-3.60 (m, 2H), 3.45 (m, 1H), 3.35 (m, 2H), 3.15 (m, 1H), 3.11 (s, 3H), 2.88-3.12 (m, 1H), 2.42 (m, 2H), 2.36 (m, 2H), 1.68 (m, 4H). MS-ESI (m/z): [M+H]⁺ calculated for C₂₄H₃₆ClN₄O₁₀⁺, 575.2; observed, 575.3. [M+Na]⁺ calculated for C₂₄H₃₅ClN₄NaO₁₀⁺, 597.2; observed, 597.2. HRMS-ESI (m/z): [M+Na]⁺ calculated for C₂₄H₃₅ClN₄NaO₁₀, 597.1934; observed, 597.1938.

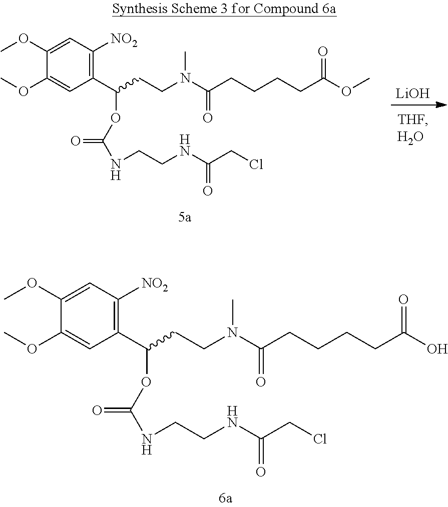

### 1-Chloro-9.(4,5-dimethoxy-2-nitrophenyl)-12-methyl-2,7,13-trioxo-8-oxa-3,6,12-triazaoctadecan-18-oic acid (6a)

Compound 5a (18.6 mg, 0.0323 mmol) was dissolved in THF (100 μL), and cooled to 0° C. To this solution, LiOH (1.49 mg, 0.0356 mmol) aqueous solution (133 μL) was added slowly, and allowed to stir at room temperature for 2 h, at this time point, the reaction mixture was diluted with EtOAc, washed once with 2 M HCl (133 μL), and organic layer was dried over Na₂SO₄. Solvent was removed in vacuo to afford compound 6a as a colorless oil (16.7 mg, 93%). ¹H NMR (400 MHz, CDCl₃) δ 8.00 (m, 1H), 7.61 (br, s, 1H), 7.02 (br, s, 1H), 6.06 (m, 1H), 5.43 (m, 1H), 4.38 (m, 1H), 4.01 (s, 2H), 3.97 (s, 3H), 3.93 (s, 3H), 3.50-3.60 (m, 2H), 3.45 (m, 1H), 3.30-3.42 (m, 2H), 3.21 (m, 1H), 3.12 (s, 3H), 3.08 (m, 1H), 2.48 (m, 2H), 2.38 (m, 2H), 1.70 (m, 4H). MS-ESI (m/z): [M+H]⁺ calculated for C₂₃H₃₄ClN₄O₁₀⁺, 561.2: observed, 561.3. [M+Na]⁺ calculated for C₂₃H₃₃ClN₄NaO₁₀⁺, 583.2; observed, 583.2. HRMS-ESI (m/z): [M+Na]⁺ calculated for C₂₃H₃₃ClN₄NaO₁₀, 583.1777; observed, 583.1776.

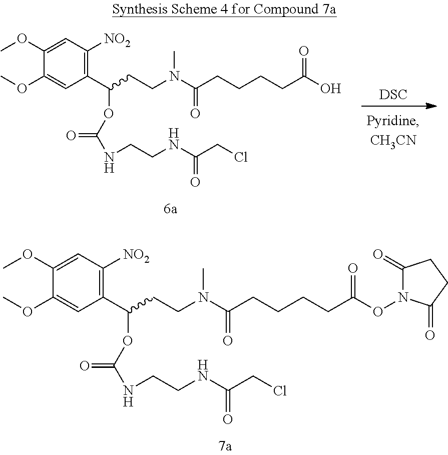

### 2,5-dioxopyrrolidin-1-yl 1-chloro-9-(4,5-dimethoxy-2-nitrophenyl)-12-methyl-2,7,13-trioxo-8-oxa-3,6,12-triazaoctadecan-18-oate (7a)

Compound 6a (16.7 mg, 0.0298 mmol), DSC (30.0 mg, 0.117 mmol) and pyridine (25.6 μL, 0.319 mmol) were dissolved in CH₃CN (350 μL) and reacted at room temperature for 16 h. Solvent was then removed in vacuo. The remaining residue was dissolved in EtOAc, washed once with 0.1 M aq. HCl, washed once with saturated aq. NaHCO₃ and dried over anhydrous Na₂SO₄. Solvent was removed in vacuo, and the residue was purified by SiO₂ column chromatography (CHCl₃/acetone, stepwise gradient from 1/0 to 1/1) to yield 7a as a colorless oil (13.9 mg, 68%). ¹H NMR (400 MHz, CDCl₃) δ 8.12 (m, 1H), 7.62 (br, s, 1H), 7.01 (br, s, 1H), 6.06 (m, 1H), 5.23 (m, 1H), 4.37 (m, 1H), 4.02 (s, 2H), 3.96 (s, 3H), 3.93 (s, 3H), 3.50-3.57 (m, 2H), 3.38 (m, 1H), 3.28-3.40 (m, 2H), 3.18 (m, 1H), 3.11 (s, 3H), 3.03 (m, 1H), 2.80-2.88 (m, 4H), 2.65 (m, 2H), 2.44 (m, 2H), 1.70-1.88 (m, 4H). MS-ESI (m/z): [M+H]⁺ calculated for C₂₇H₃₇ClN₅O₁₂⁺, 658.2: observed, 658.4. [M+Na]⁺ calculated for C₂₇H₃₆ClN₅NaO₁₂⁺, 680.2; observed, 680.4. HRMS-ESI (m/z): [M+Na]⁺ calculated for C₂₇H₃₆ClN₅NaO₁₂, 680.1941; observed, 680.1945. Product 7a may be abbreviated as:

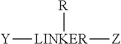

wherein the R group (e.g. DMNB and equivalent structures) is a photoactive group utilized in the process of photocleavage and the X and Z groups are suitable functional groups utilized for attachment to a morpholino to generate a cyclic structure.

Compound 2b was synthesized according to reported procedure^{1,2} from a commercially available compound, 3-(methylamino)propan-1-ol.

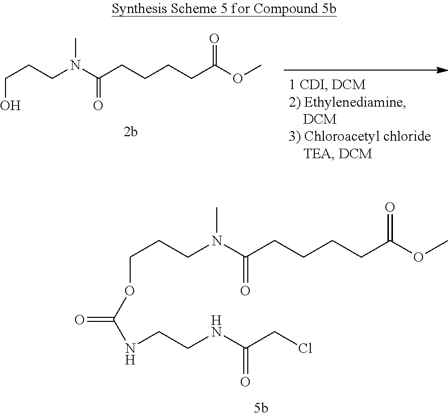 [SMILES: COC(=O)CCCCC(=O)N(C)CCCO ; COC(=O)CCCCC(=O)N(C)CCCOC(=O)NCCNC(=O)CCl]

### Methyl 1-chloro-12-methyl-2,7,13-trioxo-8-oxa-3,6,12-triazaoctadecan-18-oate (5b)

Compound 2b (67.3 mg, 0.291 mmol) was dissolved in anhydrous DCM (1.00 mL) and added to 1,1′-carbonyl diimidazole (118 mg, 0.728 mmol) in anhydrous DCM (1.30 mL). The reaction mixture was stirred for 3 h at room temperature under nitrogen, diluted with CHCl₃, washed two times with water, and dried over anhydrous MgSO₄. Solvent was removed in vacuo to yield crude imidazole carbomate as a yellow gum 3b (71.5 mg, 0.222 mmol).

The imidazole carbamate 3b (71.5 mg, 0.222 mmol) was dissolved in anhydrous DCM (500 μL), and the solution was cooled to 0° C. Ethylenediamine (45.0 μL, 0.673 mmol) was added, and the reaction mixture was stirred for 2 h at room temperature under nitrogen. Solvent was removed in vacuo to yield crude amine 4b as a yellow oil (63.7 mg, 0.201 mmol). MS-ESI (m/z): [M+H]⁺ calculated for C₁₄H₂₈N₃O₅⁺, 318.2; observed, 318.3. Without further purification, compound 4b (63.7 mg, 0.201 mmol) was dissolved in anhydrous DCM (3 mL) and triethylamine (140.7 μL, 1.01 mmol) and cooled to 0° C. 2-Chloroacetyl chloride (32.0 μL, 0.402 mmol) dissolved in anhydrous was added slowly to this solution. The mixture was allowed to stir at room temperature for 20 min, at which time, 5% saturated aq. NaHCO₃ was added. Organic layer was dried over anhydrous MgSO₄. Solvent was removed in vacuo, and the residue was purified by SiO₂ column chromatography (CHCl₃/acetone, stepwise gradient from 1/0 to 1/1) to yield 5a as a colorless oil (56.8 mg, 50% from 3b). ¹H NMR (400 MHz, CDCl₃) δ 7.42 (m, 1H), 5.41 (m, 1H), 4.12 (m, 2H), 4.04 (s, 2H), 3.67 (s, 3H), 3.45 (m, 2H), 3.42 (m, 2H), 3.34 (m, 2H), 2.99 (s, 3H), 2.28-2.39 (m, 4H), 1.85 (m, 2H), 1.62 (m, 4H). MS-ESI (m/z): [M+H]⁺ calculated for C₁₆H₂₉ClN₃O₆⁺, 394.2; observed, 394.3. [M+Na]⁺ calculated for C₁₆H₂₈ClN₃NaO₆⁺, 416.2; observed, 416.3. HRMS-ESI (m/z): [M+Na]calculated for C₁₆H₂₈ClN₃NaO₆, 416.1559; observed, 416.1550.

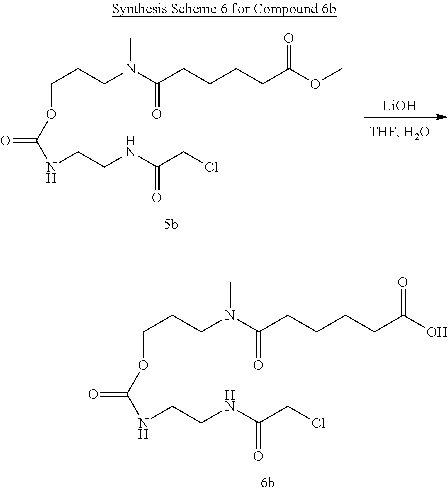 [SMILES: COC(=O)CCCCC(=O)N(C)CCCOC(=O)NCCNC(=O)CCl ; "LiOH" ; CN(CCCOC(=O)NCCNC(=O)CCl)C(=O)CCCCC(=O)O]

### 1-Chloro-12-methyl-2,7,13-trioxo-8-oxa-3,6,12-triazaoctadecan-18-oic acid (6b)

Compound 5b (40.8 mg, 0.104 mmol) was dissolved in THF (400 μL), and cooled to 0° C. To this solution, LiOH (4.79 mg, 0.114 mmol) aqueous solution (333 μL) was added slowly, and allowed to stir at room temperature for 2.5 h, at this time point, the reaction mixture was diluted with EtOAc, washed once with 2 M HCl (533 μL), and organic layer was dried over Na₂SO₄. Solvent was removed in vacuo to afford compound 6a as a colorless oil (19.5 mg, 49%). ¹H NMR (400 MHz, CDCl₃) δ 7.42 (m, 1H), 5.29 (m, 2H), 4.08 (m, 2H), 4.06 (s, 2H), 3.47 (m, 2H), 3.32-3.43 (m, 4H), 3.00 (s, 3H), 2.32-2.41 (m, 4H), 1.82-1.93 (m, 2H), 1.67-1.73 (m, 4H). MS-ESI (m/z): [M+H]⁺ calculated for C₁₅H₂₇ClN₃O₆⁺, 380.2: observed, 380.2. [M+Na]⁺ calculated for C₁₅H₂₆ClN₃NaO₆⁺, 402.1; observed, 402.2. HRMS-ESI (m/z): [M+Na]⁺ calculated for C₁₅H₂₆ClN₃NaO₆, 402.1402; observed, 402.1401.

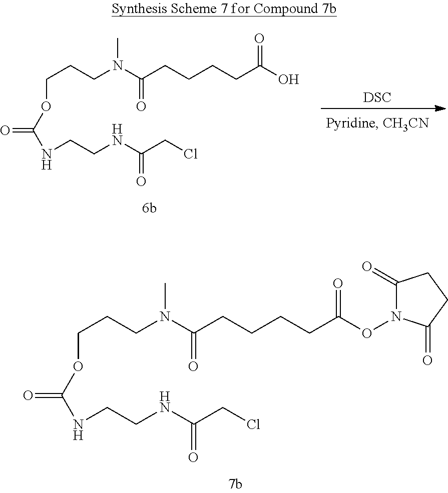 [SMILES: CN(CCCOC(=O)NCCNC(=O)CCl)C(=O)CCCCC(=O)O ; "DSC" ; CN(CCCOC(=O)NCCNC(=O)CCl)C(=O)CCCCC(=O)ON1C(=O)CCC1=O]

### 2,5-Dioxopyrrolidin-1-yl 1-chloro-12-methyl-2,7,13-trioxo-8-oxa-3,6,12-triazaoctadecan-18-oate (7b)

Compound 6b (19.5 mg, 0.0513 mmol), DSC (65.3 mg, 0.255 mmol) and pyridine (45.0 μL, 0.514 mmol) were dissolved in CH₃CN (700 μL) and reacted at room temperature for 16 h. Solvent was then removed in vacuo. The remaining residue was dissolved in EtOAc, washed once with 0.1 M aq. HCl, washed once with saturated aq. NaHCO₃ and dried over anhydrous Na₂SO₄. Solvent was removed in vacuo to yield 7b as a colorless oil (19.5 mg, 79%). ¹H NMR (400 MHz, CDCl₃) δ 7.42 (m, 1H), 5.72 (m, 1H), 4.12 (m, 2H), 4.06 (s, 2H), 3.41-3.52 (m, 4H), 3.36 (m, 2H), 3.01 (s, 3H), 2.82-2.89 (m, 4H), 2.66 (m, 2H), 2.37-2.4 (m, 2H), 1.87-1.93 (m, 2H), 1.69-1.88 (m, 4H). MS-ESI (m/z): [M+H]⁺ calculated for C₁₉H₃₀ClN₄O₈⁺, 477.2: observed, 477. [M+Na]⁺ calculated for C₁₉H₂₉ClN₄NaO₈⁺, 499.2; observed, 499.3. HRMS-ESI (m/z): [M+Na]⁺ calculated for C₁₉H₂₉ClN₄NaO₈, 499.1566; observed, 499.1558.

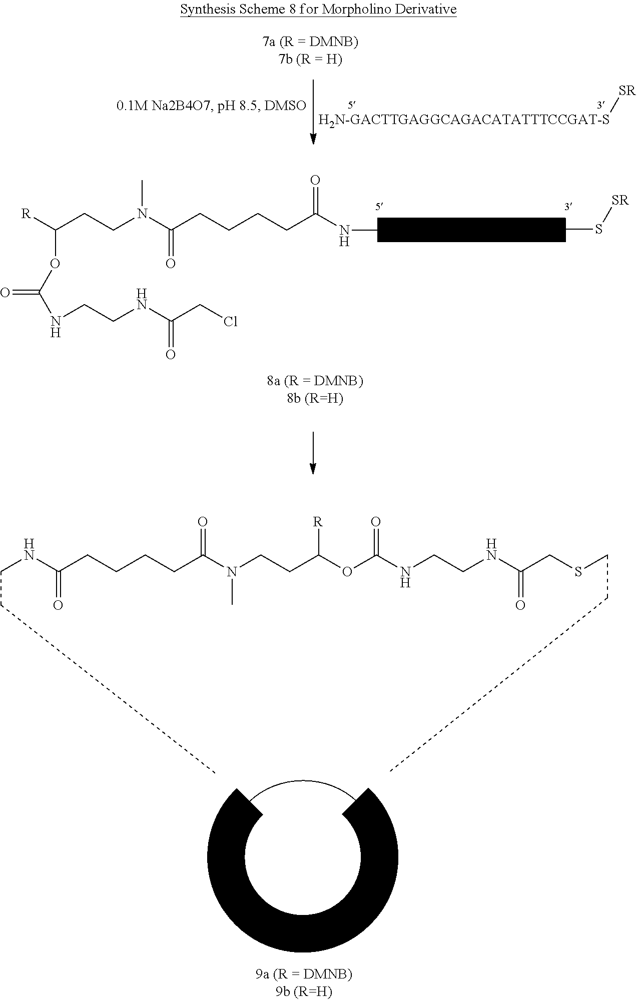

### Reaction of bifunctional linker (7a) with 5′-amine and 3′-disulfide morpholino

An MO oligomer (5′-GACTTGAGGCAGACATATTTCCGAT-3′) with 5′-amine and 3′-disulfide was purchased from Gene-Tools, LLC. This MO (56.0 nmol) was dissolved in 0.1 M Na₂B₄O₇ pH8.5 (100 μL). The MO solution was added by 15.0 μL DMSO solution of photocleavable linker (7a, 5 equiv. to MO). The reaction was shaken overnight in the dark. The reaction mixture was subjected to Nap-5 column, and lyophilized The resulting white solid was dissolved in 200 μL water, and added by 2 μL of acetic acid. The acidified solution was washed with chloroform (3×200 μL), washed with EtOAc (2×200 μL), and neutralized with 10% ammonium hydroxide aqueous solution. The solution was lyophilized to dryness, affording the conjugated product (8a, 95%) as a white solid. MS-ESI (m/z): [M+H]⁺ calculated for 8a C₃₄₀H₅₃₀ClN₁₅₇O₁₁₄P₂₅S₂, 9515; observed 9514. Product 8a may be abbreviated as:

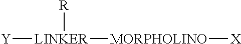

wherein the R group (e.g. DMNB and equivalent structures) is a photoactive group utilized in the process of photocleavage and the X and Y groups are suitable functional groups that react with one another for cyclization.

The same procedure was used for the reaction using nonphotocleavable linker. The synthetic yield of 8b was 76%. MS-ESI (m/z): [M+H]⁺ calculated for 8a C₃₃₂H₅₂₂ClN₁₅₆O₁₁₀P₂₅S₂, 9333; observed 9334.

Reduction and Cyclization

Immobilized TCEP disulfide reducing gel (100 μL) was purchased from Pierce Biotechnology, Inc. The gel was washed three times with 0.1 M Tris buffer pH8.4 (3×100 μL) in a centrifuge filter tube. The conjugated product (8a, 38.0 nmol) was dissolved in 0.1 M Tris buffer pH8.4 (100 μL), and added to the washed gel. The reaction was shaken for 10 h in the dark. The supernatant was collected by centrifuge at 1000 rpm for 30 sec. The gel slurry was washed by 0.1 M Tris buffer pH8.4 (3×100 μL) and washed fractions were combined to the supernatant. This mixture was subjected to Nap-5 column, and lyophilized to afford a cyclized product (9a, 84%). MS-ESI (m/z): [M+H]⁺ calculated for 9a C₃₃₅H₅₂₂N₁₅₄O₁₁₃P₂₅S, 9361; observed 9362. Product 9a may be abbreviated as:

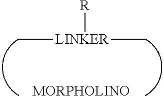

wherein the R group (e.g. DMNB and equivalent structures) is a photoactive group utilized in the process of photocleavage of the cyclic caged morpholino.

The same procedure was used for the preparation of cyclic MOs using other linkers.

Non-Photocleavable ntla MO (9b).

The same procedure was used for the preparation of cyclic MOs using non-photocleavable linker. The synthetic yield of 9b was 72%. MS-ESI (m/z): [M+H]⁺ calculated for 9b C₃₂₇H₅₁₄N₁₅₃O₁₀₉P₂₅S, 9179; observed 9179.

### 23-mer (5′-GACTTGAGGCAGACATATTTCCG-3′) cyclic ntla MO (9c)

Synthetic procedures identical to those for 25-mer ntl cMOs 9a were utilized. The synthetic yields of 8c (from MO) and 9c (from 8c) were 69% and 92%, respectively. MS-ESI (M/Z). [M+H]⁺ calculated for 8c C₃₁₆H₄₉₃ClN₁₄₆O₁₀₆P₂₃S₂, 8845; observed 8845. MS-ESI (m/z): [M+H]⁺ calculated for 9c C₃₁₂H₄₈₅N₁₄₅O₁₀₅P₂₃S, 8691; observed 8692.

### 21-mer (5′-CTTGAGGCAGACATATTTCCG-3′) cyclic ntla MO (9d)

Synthetic procedures identical to those for 25-mer ntl cMOs 9a were utilized. The synthetic yields of 8d (from MO) and 9d (from 8c) were 78% and 90%, respectively. MS-ESI (m/z): [M+H]⁺ calculated for 8d C₂₉₂H₄₅₇ClN₁₃₂O₉₉P₂₁S₂, 8151; observed 8151. MS-ESI (m/z): [M+H]⁺ calculated for 9d C₂₈₈H₄₄₉N₁₃₁O₉₈P₂₁S, 7997; observed 7997.

### 25+3-mer (5′-ATCGACTTGAGGCAGACATATTTCCGAT-3′) cyclic ntla MO (9e)

Synthetic procedures identical to those for 25-mer ntl cMOs 9a were utilized. The synthetic yields of 8e (from MO) and 9e (from 8e) were 90% and 87%, respectively. MS-ESI (m/z): [M+H]⁺ calculated for 8e C₃₇₅H₅₈₅ClN₁₇₃O₁₂₆P₂₈S₂, 10499; observed 10498. MS-ESI (m/z): [M+H]⁺ calculated for 9e O₃₇₁H₅₇₇N¹⁷²O₁₂₅P₂₈S, 10345; observed 10345.

### 24+4-mer (5′-TCGGGACTTGAGGCAGACATATTTCCGA-3′) cyclic ntla MO (9f)

Synthetic procedures identical to those for 25-mer ntl cMOs 9a were utilized with modifications of a final purification. Mixture of supernatant and washed fractions from TCEP resin was applied to 100 μL of SulfoLink Coupling Resin (Thermo Scientific) in a centrifuge filter tube. The resin was shaken for 15 minutes at room temperature and stood upright without shaking for 30 minutes at room temperature. The supernatant was collected by centrifuge at 1000 rpm for 30 sec. The gel slurry was washed by 0.1 M Tris buffer pH8.4 (3×100 μL) and washed fractions were combined to the supernatant. This mixture was subjected to Nap-5 column, and lyophilized to afford a cyclized product (12%, 2 steps from MO). MS-ESI (m/z): [M+H]⁺ calculated for 9f C₃₇₁H₅₇₆N₁₇₅O₁₂₅P₂₈S, 10386; observed 10387.

### 25-mer (5′-CCAACACAGTGTCCATTTTTTGTGC-3′) cyclic ptf1a cMO (9g)

Synthetic procedure identical to that for 25-mer cyclic ntla cMO 9a was utilized. The synthetic yields of 8g (from MO) and 9g (from 8g) were 62% and 51%, respectively. MS-ESI (m/z): [M+H]⁺ calculated for 8g C₃₃₈H₅₃₃ClN₁₄₇O₁₁₈P₂₅S₂, 9418; observed 9417. MS-ESI (m/z): [M+H]⁺ calculated for 9g C₃₃₄H₅₂₄N₁₄₆O₁₁₇P₂₅S, 9263; observed 9263.

Synthesis of Hairpin cMOs.

Hairpin cMOs against ntla and ptf1a were synthesized according to reported procedure² from commercially available end-modified MOs.

Hairpin ntla cMO (11).

MO oligomer (5′-GACTTGAGGCAGACATATTTCCGAT-3′) and non-fluorescein labeled inhibitor MO (iMO) oligomer (5′-GCCTCAAGTC-3′) were used. Final product was recovered as a white solid (7.08 nmol, 7% overall). MS-ESI (m/z): [M+H]⁺ calculated for 11 C₄₆₄H₇₂₂N₂₁₈O₁₅₆P₃₅ 12934. found, 12935.

Hairpin ptf1a cMO (12).

MO oligomer (5′-CCAACACAGTGTCCATTTTTTGTGC-3′) and non-fluorescein labeled iMO oligomer (5′-CACTGTGTTGG-3′) were used. Final product was recovered as a white solid (4.34 nmol, 4% overall). MS-ESI (m/z): [M+H]⁺ calculated for 12 C₄₇₆H₇₄₄N₂₁₃O₁₆₇P₃₆13237. found, 13240.

### Examples

### Biological Testing

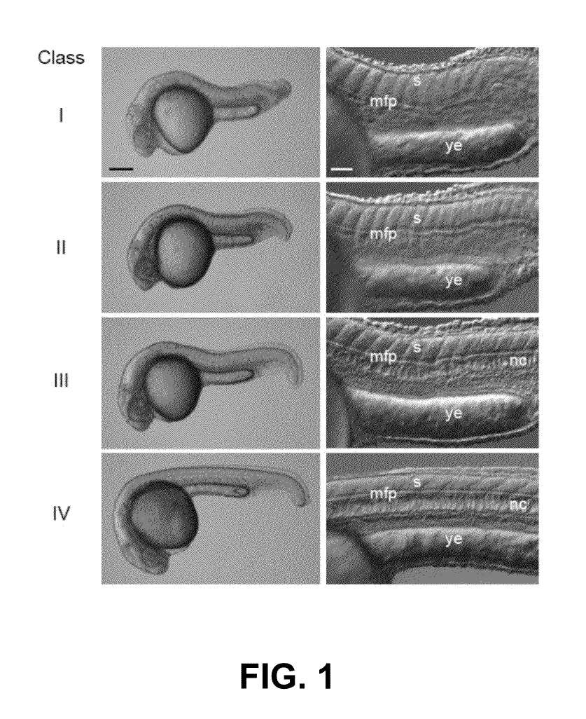

Comparison of Cyclic and Hairpin Ntla cMO Efficacies as See in FIGS. 1-3.

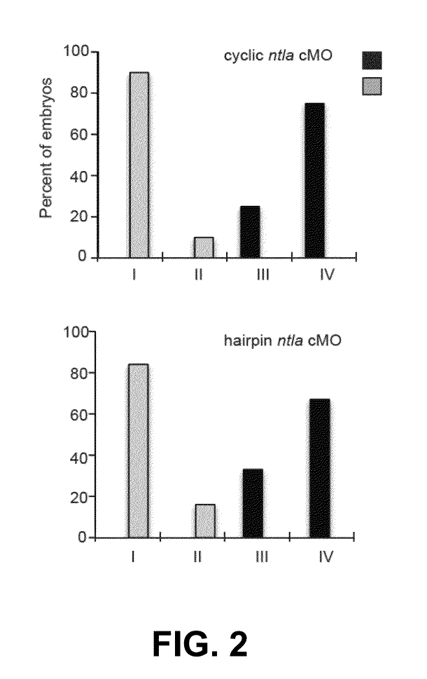

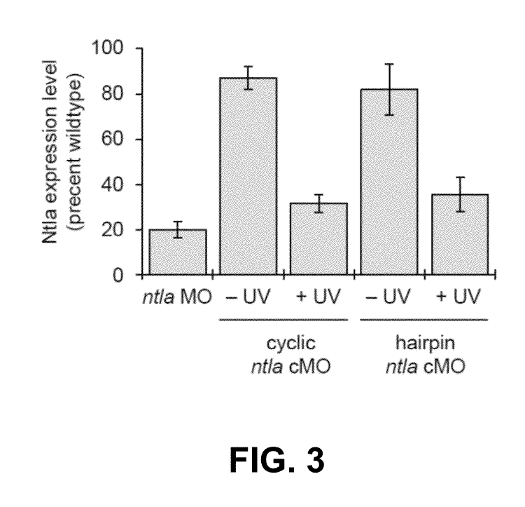

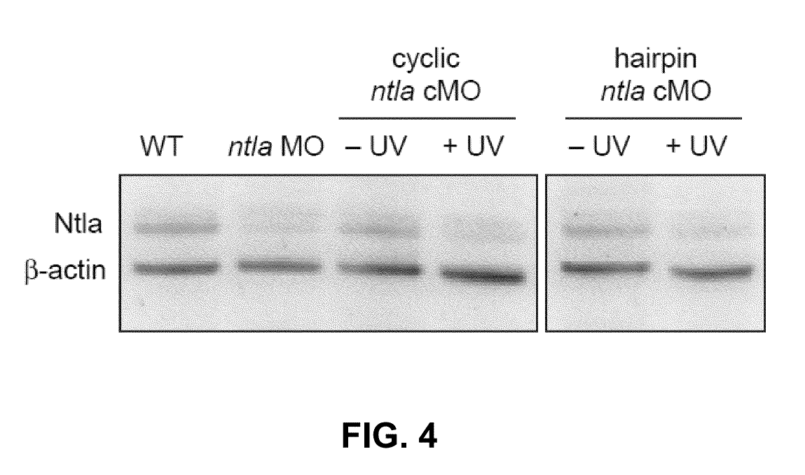

No tail-a (Ntla) is a T-box transcription factor required for notochord development, and loss of Ntla function results in the complete absence of notochord tissues, ectopic medial floor plate, and U-shaped somites. Using these morphological criteria, the efficacies of cyclic and hairpin ntla cMOs have been compared. FIG. 1 shows the classification of ntla mutant phenotypes according to morphological cues. Somitic (s), medial floor plate (mfp), notochord (nc), and yolk extension (ye) tissues are labeled. Scale bars are: left panels, 200 μm and right panels, 50 μm. Bright-field (left) and differential interference contrast (right) images of 1-day-post-fertilization (dpf) embryos are shown, anterior to the left and dorsal up. FIG. 2 presents the distribution of phenotypes for cyclic and hairpin cMOs at a dose of 115 fmol/embryo with and without global UV photoactivation at 3 hours post fertilization (hpf). Between 13 and 24 embryos were analyzed for each experimental condition. FIG. 3 depicts the measurement of Ntla protein levels in 10-hpf embryos injected with a conventional ntla MO, a cyclic ntla cMO, or a hairpin ntla cMO at the one-cell stage and then either cultured in the dark or globally UV irradiated at 3 hpf. Data are the average Ntla protein levels in at least 7 independent experiments, each utilizing 20 embryos per experimental condition and normalized with respect to β-actin (error bars represent s.e.m.). FIG. 4 shows a representative western blot for the experimental conditions described above. These results demonstrate that cyclic and hairpin cMOs can have similar efficacies when used to study gene function within the first day of zebrafish development.

Comparison of Cyclic and Hairpin ptf1a cMO Efficacies as Seen in FIGS. 4-7.

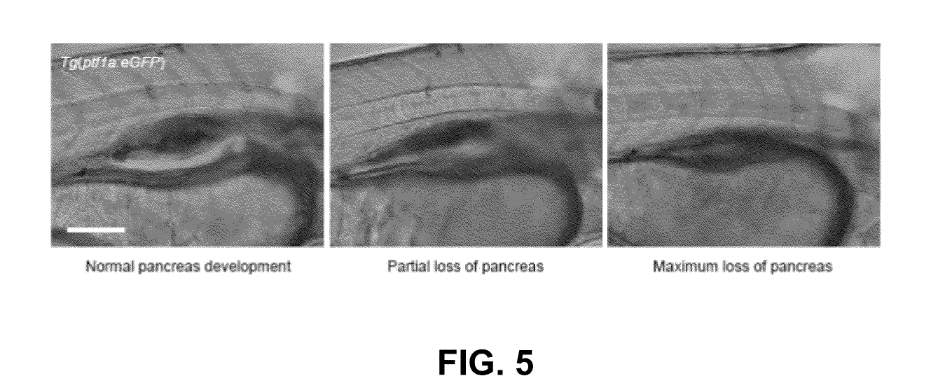

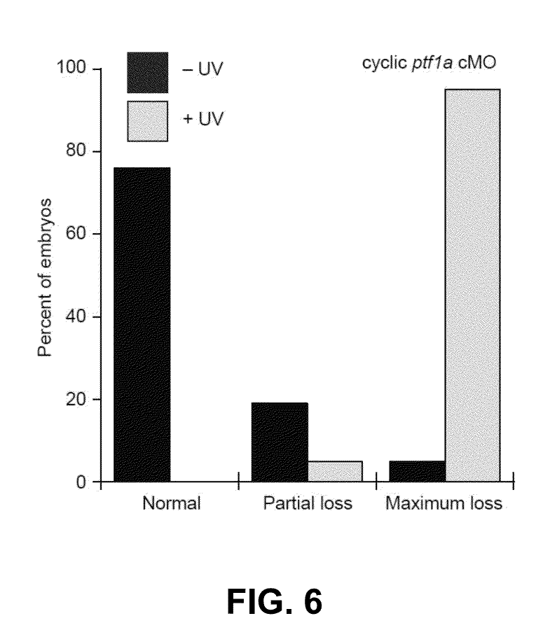

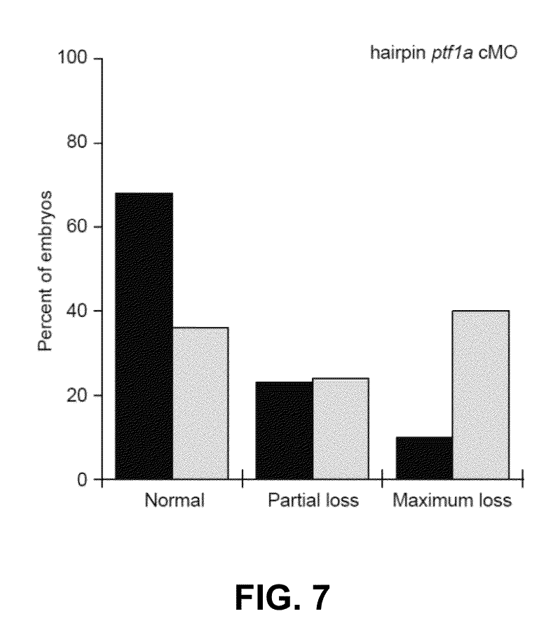

Pancreas-specific transcription factor 1 alpha (Ptf1a) is required for pancreas development, which can be visualized in transgenic zebrafish [Tg(ptf1a:eGFP)]. Loss of Ptf1a function prevents the formation of ptf/a-positive exocrine cells, leading a loss of enhanced green fluorescent protein (eGFP) expression within the pancreatic field. FIG. 5 shows the classifications of ptf1a mutant phenotypes according to pancreas-specific eGFP expression in the transgenic zebrafish. Fluorescence micrographs of 3-dpf larvae are shown, anterior to the right and dorsal up, with the scale at bar: 100 μm. FIGS. 6 and 7 show the distributions of phenotypes for cyclic (FIG. 6) and hairpin (FIG. 7) cMOs at a dose of 230 fmol/embryo with and without global UV photoactivation at 3 hours post fertilization (hpf). Between 21 and 31 embryos were analyzed for each experimental condition. These results indicate that cyclic cMOs can be significantly more effective than hairpin cMOs when studying gene function at later stages of development, such as in zebrafish larvae.

Embodiments of the subject invention include a bifunctional and photocleavable linker for cyclizing a morpholino-based oligonucleotide, comprising the formula:

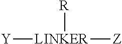

wherein the Linker is photoactive group derivatized spacer molecule that presents two functional groups for cyclizing a selected morpholino and wherein the R group is the photoactive group utilized in the process of photocleavage and the X and Z groups are functional groups utilized for attachment to a morpholino to generate a cyclic structure.

An additional embodiment of the subject invention is a bifunctional and photocleavable linker for cyclizing a morpholino-based oligonucleotide, comprising the formula:

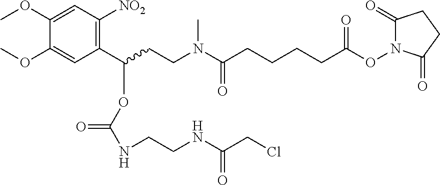 [SMILES: COc1cc(C(CCN(C)C(=O)CCCCC(=O)ON2C(=O)CCC2=O)OC(=O)NCCNC(=O)CCl)c([N+](=O)[O-])cc1OC]

Another embodiment of the subject invention is a morpholino containing compound having a photocleavable linker comprising the formula:

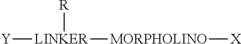

wherein the R group (e.g. DMNB and equivalent structures) is a photoactive group utilized in the process of photocleavage and the X and Y groups are suitable functional groups that react with one another for cyclization. For exemplary purposes only and not by way of limitation, the morpholino may be selected from a group consisting of

5′-CCAACACAGTGTCCATTTTTTGTGC-3′,
5′-GACTTGAGGCAGACATATTTCCGAT-3′,
5′-CTTGAGGCAGACATATTTCCG-3′,
5′-ATCGACTTGAGGCAGACATATTTCCGAT-3′,
and
5′-TCGGGACTTGAGGCAGACATATTTCCGA-3′.

A further embodiment of the subject invention is a cyclic caged morpholino having a photocleavable linker comprising the formula:

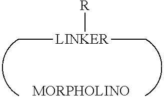

wherein the Linker is a spacer molecule and wherein the R group is a photoactive group utilized in the process of photocleavage of the cyclic caged morpholino, wherein, for exemplary purposes only and not by way of limitation, the morpholino is selected from a group consisting of

5′-CCAACACAGTGTCCATTTTTTGTGC-3′,
5′-GACTTGAGGCAGACATATTTCCGAT-3′,
5′-CTTGAGGCAGACATATTTCCG-3′,
5′-ATCGACTTGAGGCAGACATATTTCCGAT-3′,
and
5′-TCGGGACTTGAGGCAGACATATTTCCGA-3′.

Yet another embodiment of the subject invention is a cyclic caged morpholino having a photocleavable linker comprising the formula:

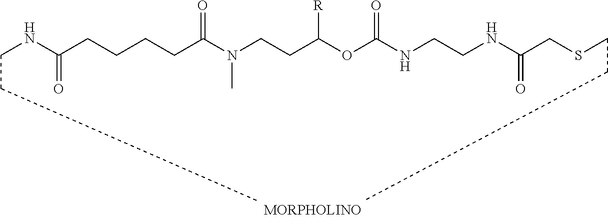

wherein the R group is a photoactive group utilized in the process of photocleavage of the cyclic caged morpholino, wherein, for exemplary purposes only and not by way of limitation, the morpholino is selected from a group consisting of

5′-CCAACACAGTGTCCATTTTTTGTGC-3′,
5′-GACTTGAGGCAGACATATTTCCGAT-3′,
5′-CTTGAGGCAGACATATTTCCG-3′,
5′-ATCGACTTGAGGCAGACATATTTCCGAT-3′,
and
′-TCGGGACTTGAGGCAGACATATTTCCGA-3′.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A bifunctional and photocleavable linker for cyclizing a morpholino-based oligonucleotide, comprising the formula: 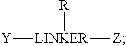
wherein said Linker is photoactive group derivatized spacer molecule that presents two functional groups for cyclizing a selected morpholino; and
wherein said R group is said photoactive group utilized in the process of photocleavage and said X and Z groups are functional groups utilized for attachment to a morpholino to generate a cyclic structure.

2. A bifunctional and photocleavable linker for cyclizing a morpholino-based oligonucleotide, comprising the formula:

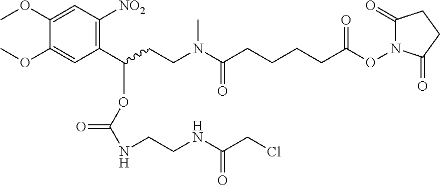 [SMILES: COc1cc(C(CCN(C)C(=O)CCCCC(=O)ON2C(=O)CCC2=O)OC(=O)NCCNC(=O)CCl)c([N+](=O)[O-])cc1OC]

3. A morpholino containing compound having a photocleavable linker comprising the formula: 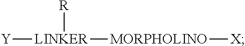
wherein said R group is a photoactive group utilized in the process of photocleavage and said X and Y groups are functional groups that react with one another for cyclization.

4. A morpholino containing compound having a photocleavable linker according to claim 3, wherein said morpholino is selected from a group consisting of
5′-CCAACACAGTGTCCATTTTTTGTGC-3′,
5′-GACTTGAGGCAGACATATTTCCGAT-3′,
5′-CTTGAGGCAGACATATTTCCG-3′,
5′-ATCGACTTGAGGCAGACATATTTCCGAT-3′,
and
5′-TCGGGACTTGAGGCAGACATATTTCCGA-3′.

5. A morpholino containing compound having a photocleavable linker comprising the formula: 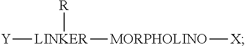
wherein said R group is a photoactive group utilized in the process of photocleavage and said X and Y groups are functional groups that react with one another for cyclization; and
wherein said morpholino is selected from a group consisting of
5′-CCAACACAGTGTCCATTTTTTGTGC-3′,
5′-GACTTGAGGCAGACATATTTCCGAT-3′,
5′-CTTGAGGCAGACATATTTCCG-3′,
5′-ATCGACTTGAGGCAGACATATTTCCGAT-3′,
and
5′-TCGGGACTTGAGGCAGACATATTTCCGA-3′.

6. A cyclic caged morpholino having a photocleavable linker comprising the formula: 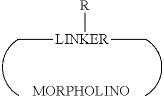
wherein said Linker is a spacer molecule; and
wherein said R group is a photoactive group utilized in the process of photocleavage of the cyclic caged morpholino.

7. A cyclic caged morpholino having a photocleavable linker according to claim 6, wherein said morpholino is selected from a group consisting of
5′-CCAACACAGTGTCCATTTTTTGTGC-3′,
5′-GACTTGAGGCAGACATATTTCCGAT-3′,
5′-CTTGAGGCAGACATATTTCCG-3′,
5′-ATCGACTTGAGGCAGACATATTTCCGAT-3′,
and
5′-TCGGGACTTGAGGCAGACATATTTCCGA-3′.

8. A cyclic caged morpholino having a photocleavable linker comprising the formula: 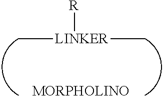
wherein said Linker is a spacer molecule;
wherein said R group is a photoactive group utilized in the process of photocleavage of the cyclic caged morpholino; and
wherein said morpholino is selected from a group consisting of
5′-CCAACACAGTGTCCATTTTTTGTGC-3′,
5′-GACTTGAGGCAGACATATTTCCGAT-3′,
5′-CTTGAGGCAGACATATTTCCG-3′,
5′-ATCGACTTGAGGCAGACATATTTCCGAT-3′,
and
5'-TCGGGACTTGAGGCAGACATATTTCCGA-3′.

9. A cyclic caged morpholino having a photocleavable linker comprising the formula: 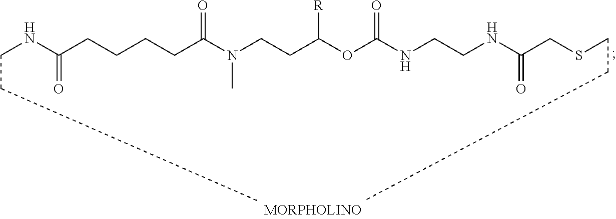
wherein the R group is a photoactive group utilized in the process of photocleavage of the cyclic caged morpholino.

10. A cyclic caged morpholino having a photocleavable linker according to claim 9, wherein said morpholino is selected from a group consisting of
5′-CCAACACAGTGTCCATTTTTTGTGC-3′,
5′-GACTTGAGGCAGACATATTTCCGAT-3′,
5′-CTTGAGGCAGACATATTTCCG-3′,
5′-ATCGACTTGAGGCAGACATATTTCCGAT-3′,
and
5′-TCGGGACTTGAGGCAGACATATTTCCGA-3′.

11. A cyclic caged morpholino having a photocleavable linker comprising the formula: 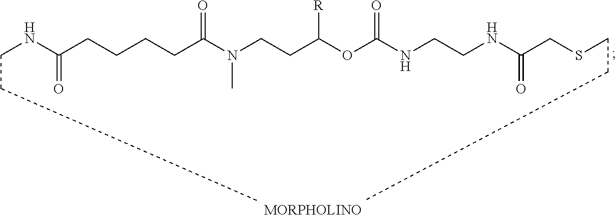
wherein the R group is a photoactive group utilized in the process of photocleavage of the cyclic caged morpholino; and
wherein said morpholino is selected from a group consisting of
5′-CCAACACAGTGTCCATTTTTTGTGC-3′,
5′-GACTTGAGGCAGACATATTTCCGAT-3′,
5′-CTTGAGGCAGACATATTTCCG-3′,
5′-ATCGACTTGAGGCAGACATATTTCCGAT-3′,
and
5′-TCGGGACTTGAGGCAGACATATTTCCGA-3′.
